(19) United States
(12) Patent Application Publication
(10) Pub. No.: US 2024/0418718 A1
(43) Pub. Date: Dec. 19, 2024

(54) METHOD AND KIT FOR IMMUNOTYPING OF T LYMPHOCYTE DEVELOPMENT SUBGROUPS

(71) Applicant: CHILDREN’S HOSPITAL OF CHONGQING MEDICAL UNIVERSITY, Chongqing (CN)

(72) Inventors: Yao ZHAO, Chongqing (CN); Linlin NIU, Chongqing (CN); Bingqian TAN, Chongqing (CN); Yuting YANG, Chongqing (CN)

(73) Assignee: CHILDREN’S HOSPITAL OF CHONGQING MEDICAL UNIVERSITY, Chongqing (CN)

(21) Appl. No.: 18/721,917

(22) PCT Filed: Mar. 3, 2022
(86) PCT No.: PCT/CN2022/078965

§ 371 Date: Jun. 20, 2024

(30) Foreign Application Priority Data
Dec. 22, 2021 (CN) 202111584011.5

(51) Int. Cl. G01N 33/569

(52) U.S. Cl. CPC G01N 33/56972

(57) ABSTRACT

A method and kit for the immunotyping of T lymphocyte development subgroups. The method for the immunotyping of T lymphocyte development subgroups comprises: taking antibodies with different fluorescence labels, mixing same with a to-be-detected sample, performing incubating, and then performing detection by means of flow cytometry to obtain detection data, wherein the antibodies comprise: an anti-CD45 antibody, an anti-CD3 antibody, an anti-CD4 antibody, an anti-CD8 antibody, an anti-CD25 antibody, an anti-CD127 antibody, an anti-CD45RO antibody, an anti-CD28 antibody, an anti-CD197 antibody and an anti-CD95 antibody. According to the present invention, more comprehensive immunotyping of T lymphocyte development subgroups is achieved, few to-be-detected samples are required, operation is easy, the required time is shorted, and the accuracy is high.

This application claims the priority of Chinese Patent Application No. 202111584011.5, filed with the China National Intellectual Property Administration on 22 Dec. 2021, and titled with “METHOD AND KIT FOR IMMUNOTYPING OF T LYMPHOCYTE DEVELOPMENT SUBGROUPS”, the disclosure of which is hereby incorporated by reference in its entirety.

### FIELD

The present invention relates to the field of biotechnology, and in particularly relates to an immunophenotyping method and kit for identifying a development subset of T lymphocyte.

### BACKGROUND

T lymphocytes, i.e., thymus-dependent lymphocytes, also called T cells, are an actively functional cell population that mediates cellular immune responses. T cells are derived from hematopoietic stem cells and develop in the thymus. In peripheral blood, T cells make up about 60% of the total number of lymphocytes.

T cells can be divided into several subsets of helper T cells (Th), cytotoxic T cells (Tc), and regulatory T cells (Tr) according to different functions thereof in the immune response. With the advancement of science and technology, more refined phenotypes of T cell subsets including initial T cells, memory T cells, and effector T cells can be defined based on different surface markers. T cells are responsible for immune response, homeostasis, and the establishment and maintenance of immune memory, which express a receptor with the potential to recognize multiple antigens from pathogens, tumors and the environment, maintain immune memory and have self-tolerance. T cells are also considered to be the main driver of many inflammatory and autoimmune diseases, and are essential for inducing protective immunity against pathogens or cancer.

Subsets of T lymphocytes in the blood are crucial for immune system of body. T lymphocytes make up about 60% of peripheral blood lymphocytes, and therefore CD3+ T cells correspondingly make up about 60%. However, due to technical reasons, the number of T lymphocytes detected often does not reach the total number of peripheral blood lymphocytes. Among the techniques used, the rosette assay using a monoclonal antibody has the lowest positivity rate of about 50%, while the positivity rate is higher with the fluorescence microscopy technique using a monoclonal antibody. Currently, T lymphocytes are often detected using flow cytometry. However, there is no further detection for the development of T lymphocytes. In conclusion, the above methods for detecting T cells have a narrow detection range and a complex operation, require a large amount of samples and are time-consuming. For refined immunophenotyping of T cell development subsets, a simple and time-saving method and quantitative analysis which require a small amount of sample is desirable.

### SUMMARY

In view of the above, the present invention provides an immunophenotyping method and kit for identifying a development subset of T lymphocyte. The method can achieve a more comprehensive and refined immunophenotyping and quantitative analysis for T lymphocytes, with a high efficiency, and requires a small amount of sample and a short time, which is more suitable for identification and quantitative analysis of developmental subsets of T lymphocyte.

To achieve the above object, the present invention provides the following technical solution.

The present invention provides an immunophenotyping method for identifying a development subset of T lymphocytes, comprising
mixing a composition 1 comprising antibodies labeled by different fluorescent labels with a sample, incubating, performing detection by flow cytometry, collecting detection data, and identifying the subset based on the detection data;
wherein the composition 1 comprises
an anti-CD45 antibody, an anti-CD3 antibody, an anti-CD4 antibody, an anti-CD8 antibody, an anti-CD25 antibody, an anti-CD127 antibody, an anti-CD45RO antibody, an anti-CD197 antibody, an anti-CD28 antibody, and an anti-CD95 antibody; and a criterion of the identifying is as follows:
a cell surface marker of CD3⁺CD4⁺CD25⁺CD127 indicates a Treg cell,
a cell surface marker of CD3⁺CD4⁺CD197⁺CD45RO⁻CD28⁺CD95-indicates a naive helper T cell,
a cell surface marker of CD3⁺CD4⁺CD197⁺CD45RO⁻CD28⁺CD95+ indicates a stem cell-like memory helper T cell,
a cell surface marker of CD3⁺CD4⁺CD197⁺CD45RO⁺CD28⁺CD95+ indicates a central memory helper T cell,
a cell surface marker of CD3⁺CD4⁺CD197⁻CD45RO⁺CD28-CD95+ indicates an effector memory helper T cell,
a cell surface marker of CD3⁺CD4⁺CD197⁻CD45RO⁺CD28⁺CD95+ indicates a transitional memory helper T cell,
a cell surface marker of CD3⁺CD8⁺CD197⁺CD45RO⁻CD28⁺CD95-indicates a naive cytotoxic T cell,
a cell surface marker of CD3⁺CD8⁺CD197⁺CD45RO⁻CD28⁺CD95+ indicates a stem cell-like memory cytotoxic T cell,
a cell surface marker of CD3⁺CD8⁺CD197⁺CD45RO⁺CD28⁺CD95+ indicates a central memory cytotoxic T cell,
a cell surface marker of CD3⁺CD8⁺CD197⁻CD45RO⁺CD28-CD95+ indicates an effector memory cytotoxic T cell, and
a cell surface marker of CD3⁺CD8⁺CD197⁻CD45RO⁺CD28⁺CD95+ indicates a transitional memory cytotoxic T cell.

In the present invention, an antibody used for flow cytometry may be either homologous or non-homologous to the sample, provided that it exhibits specific antigen-antibody binding to a cell surface marker of the sample.

In the present invention, “+” represents positive, i.e., an antigen is expressed on the detected cell surface;
“++” represents strong positivity, i.e., an antigen is highly expressed on the detected cell surface; and
“−” represents negative, i.e. an antigen is not expressed on the detected cell surface.

Lymphocytes, at various stages of normal differentiation and maturation and during activation, all express surface markers on their cell membrane surface that can be identified. These cell surface markers can be detected by flow cytometry using a monoclonal antibody labeled with fluorescein—as a molecular probe to further analyze the classes, sub classes, and functional properties of the cells. The present invention, according to extensive creative research and experimental validation, provides an immunophenotyping method for identifying a development subset of T cells. A criterion of the identifying is: a cell surface marker of CD3⁺CD4⁺CD25⁺CD127⁺ indicates a Treg cell; a cell surface marker of CD3⁺CD4⁺CD197⁺CD45RO⁻CD28⁺CD95⁻ indicates a naive helper T cell; a cell surface marker of CD3⁺CD4⁺CD197⁺CD45RO⁻CD28⁺CD95 indicates a stem cell-like memory helper T cell; a cell surface marker of CD3⁺CD4⁺CD197⁺CD45RO⁺CD28⁺CD95 indicates a central memory helper T cell; a cell surface marker of CD3⁺CD4⁺CD197⁻CD45RO⁺CD28⁻CD95 indicates an effector memory helper T cell; a cell surface marker of CD3⁺CD4⁺CD197⁻CD45RO⁺CD28⁺CD95 indicates a transitional memory helper T cell; a cell surface marker of CD3⁺CD8⁺CD197⁺CD45RO⁻CD28⁺CD95-indicates a naive cytotoxic T cell; a cell surface marker of CD3⁺CD8⁺CD197⁺CD45RO⁻CD28⁺CD95 indicates a stem cell-like memory cytotoxic T cell; a cell surface marker of CD3⁺CD8⁺CD197⁺CD45RO⁺CD28⁺CD95⁻ indicates a central memory cytotoxic T cell; a cell surface marker of CD3⁺CD8⁺CD197⁻CD45RO⁺CD28⁻CD95 indicates an effector memory cytotoxic T cell, and a cell surface marker of CD3⁺CD8⁺CD197⁻CD45RO⁺CD28⁺CD95⁺ indicates a transitional memory cytotoxic T cell. The present invention designs and obtains an optimal composition comprising antibodies for cell surface markers according to the cell surface markers corresponding to various cells, based on which a more comprehensive immunophenotyping analysis of T lymphocyte development subset is performed by flow cytometry. The method provided by the present invention requires a small amount of sample, has a simple operation and high accuracy, and is time-saving, which can be useful in the immunophenotyping of a development subset of T lymphocyte.

Preferably, in the immunophenotyping method for identifying a development subset of T lymphocyte provided by the present invention, a fluorescence minus one (FMO) control is further set to assess the interference of other fluorescent dyes on the target channel, which facilitates accurate determination of the threshold of positive staining, and correct setting of positive gate.

In an embodiment of the present invention, in the immunophenotyping method for identifying a development subset of T lymphocyte provided by the present invention, a fluorescent minus one (FMO) control is used as a control group. In the detection of anti-CD25 antibody labeled by BV421 (BV421-CD25), the FMO control is set with the addition of all fluorescent antibodies except BV421-CD25. In the detection of anti-CD28 antibody labeled by BV605 (BV605-CD28), the FMO control is set with the addition of all fluorescent antibodies except BV605-CD28.

In an embodiment of the present invention, the immunophenotyping method for identifying a development subset of T lymphocyte provided by the present invention particularly comprises
mixing a sample with red blood cell lysate, placing the resulting mixture in a water bath at 36.5-37.5° C. for 6-8 minutes, centrifuging to obtain a primary product, mixing the primary product with a fluorescently labeled anti-CD45 antibody, anti-CD3 antibody, anti-CD4 antibody, anti-CD8 antibody, anti-CD25 antibody, anti-CD127 antibody, anti-CD45RO antibody, anti-CD197 antibody, anti-CD28 antibody and anti-CD95 antibody, incubating at room temperature (20-25° C.) for 15-20 minutes, washing, performing detection using a flow cytometer, collecting detection data, and identifying a subset based on the detection data;
a criterion of the identifying is as follows:
a cell surface marker of CD3⁺CD4⁺CD25⁺CD127⁻ indicates a Treg cell,
a cell surface marker of CD3⁺CD4⁺CD197⁺45RO⁻CD28⁺CD95⁻ indicates a naive helper T cell,
a cell surface marker of CD3⁺CD4⁺CD197⁺CD45RO⁻CD28⁺CD95⁺ indicates a stem cell-like memory helper T cell,
a cell surface marker of CD3⁺CD4⁺CD197⁺CD45RO⁺CD28⁺CD95⁺ indicates a central memory helper T cell,
a cell surface marker of CD3⁺CD4⁺CD197-CD45RO⁺CD28-CD95⁺ indicates an effector memory helper T cell,
a cell surface marker of CD3⁺CD4⁺CD197-CD45RO⁺CD28⁺CD95⁺ indicates a transitional memory helper T cell,
a cell surface marker of CD3⁺CD8⁺CD197⁺CD45RO⁻CD28⁺CD95⁻ indicates a naive cytotoxic T cell,
a cell surface marker of CD3⁺CD8⁺CD197⁺CD45RO⁻CD28⁺CD95⁺ indicates a stem cell-like memory cytotoxic T cell,
a cell surface marker of CD3⁺CD8⁺CD197⁺CD45RO⁺CD28⁺CD95⁺ indicates a central memory cytotoxic T cell,
a cell surface marker of CD3⁺CD8⁺CD197-CD45RO⁺CD28-CD95⁺ indicates an effector memory cytotoxic T cell, and
a cell surface marker of CD3⁺CD8⁺CD197-CD45RO⁺CD28⁺CD95⁺ indicates a transitional memory cytotoxic T cell.

In the method provided by the present invention, the fluorescent labels for fluorescently labeling each antibody are not limited by the present invention, and those skilled in the art may select a suitable fluorescent label and a control method according to the actual situation.

The immunophenotyping method for identifying a development subset of T lymphocyte provided by the present invention further comprises a step of calculating the number of the T lymphocytes in the sample.

In an embodiment of the present invention, in the immunophenotyping method for identifying a development subset of T lymphocyte provided by the present invention, a step of counting the number of a development subset of the T lymphocytes in the sample comprises
detecting the total number of lymphocytes in the sample,
detecting the percentage of a development subset of the T lymphocytes to the lymphocytes in the sample; and
calculating the cell number of a development subset of the T lymphocytes in the sample.

In the method provided by the present invention, the calculating the number of the T lymphocytes in the sample is performed by multiplying the total number of the lymphocytes by the percentage of the T lymphocytes therein to obtain the number of the T lymphocytes. In the present invention, after immunophenotyping and collecting the relative number of each T cell development subset in the sample, the absolute number of each T cell subset is obtained by multiplying the absolute number of the T lymphocytes by the relative number of each T cell development subset.

In an embodiment of the present invention, a step of detecting the percentage of the T lymphocytes in the sample comprises
mixing a composition 2 comprising antibodies labeled by different fluorescent labels with the sample, incubating, performing detection by flow cytometry, collecting detection data, analyzing the detection data; and calculating the percentage of the T lymphocytes in the sample;
wherein, the composition 2 used in detecting the percentage of the T lymphocytes in the sample comprises

an anti-CD45 antibody, an anti-CD3 antibody, an anti-CD4 antibody, and an anti-CD8 antibody.

In some embodiments of the present invention, the anti-CD45 antibody is labeled by FITC as the fluorescent label in the step of detecting the percentage of the T lymphocytes in the sample in the method provided by the present invention.

In some embodiments of the present invention, the anti-CD3 antibody is labeled by APC-Cy7 as the fluorescent label in the step of detecting the percentage of the T lymphocytes in the sample in the method provided by the present invention.

In some embodiments of the present invention, the anti-CD4 antibody is labeled by Percp-Cy5.5 or BV650 as the fluorescent label in the step of detecting the percentage of the T lymphocytes in the sample in the method provided by the present invention.

In some embodiments of the present invention, the anti-CD8 antibody is labeled by BV510 as the fluorescent label in the step of detecting the percentage of the T lymphocytes in the sample in the method provided by the present invention.

In the method provided by the present invention, the step of detecting the percentage of the T lymphocytes in the sample is performed by a conventional immunophenotyping method and quantitative analysis method, which is not limited by the present invention, and those skilled in the art can select a method for detecting the percentage of the T lymphocytes in the sample according to the actual situation.

In an embodiment of the present invention, in the method provided by the present invention, the detecting the total number of lymphocytes in the sample is performed by counting using a hemacytometer or a cell counter. In the method provided by the present invention, the detecting the total number of lymphocytes in the sample is performed by a conventional method, which is not limited by the present invention, and those skilled in the art can select a method for detecting the total number of lymphocytes in the sample according to the actual situation.

The present invention further provides an immunophenotyping method for identifying a subset of T lymphocyte, comprising the step of identifying a subset of T lymphocyte provided by the present invention;
wherein, the immunophenotyping method for identifying a subset of T lymphocyte comprises:
mixing antibodies labeled by different fluorescent labels with a sample, incubating, performing detection by flow cytometry, collecting detection data, and analyzing the detection data;
wherein, the antibodies are selected from the group consisting of:
an anti-CD45 antibody, an anti-CD3 antibody, an anti-CD4 antibody, an anti-CD8 antibody, an anti-CD25 antibody, an anti-CD127 antibody, an anti-CD45RO antibody, an anti-CD197 antibody, an anti-CD28 antibody, and an anti-CD95 antibody; and
a criterion of the analyzing comprises:
a cell surface marker of CD3⁺CD4⁺CD25⁺CD127⁻ indicates a Treg cell,
a cell surface marker of CD3⁺CD4⁺CD197⁺CD45RO⁻CD28⁺CD95⁻ indicates a naive helper T cell,
a cell surface marker of CD3⁺CD4⁺CD197⁺CD45RO⁻CD28⁺CD95⁺ indicates a stem cell-like memory helper T cell,
a cell surface marker of CD3⁺CD4⁺CD197⁺CD45RO⁺CD28⁺CD95⁺ indicates a central memory helper T cell,
a cell surface marker of CD3⁺CD4⁺CD197⁻CD45RO⁺CD28⁻CD95⁺ indicates an effector memory helper T cell,
a cell surface marker of CD3⁺CD4⁺CD197⁻CD45RO⁺CD28⁺CD95⁺ indicates a transitional memory helper T cell,
a cell surface marker of CD3⁺CD8⁺CD197⁺CD45RO⁻CD28⁺CD95⁻ indicates a naive cytotoxic T cell,
a cell surface marker of CD3⁺CD8⁺CD197⁺CD45RO⁻CD28⁺CD95⁺ indicates a stem cell-like memory cytotoxic T cell,
a cell surface marker of CD3⁺CD8⁺CD197⁺CD45RO⁺CD28⁺CD95⁺ indicates a central memory cytotoxic T cell,
a cell surface marker of CD3⁺CD8⁺CD197⁻CD45RO⁺CD28⁻CD95⁺ indicates an effector memory cytotoxic T cell, and
a cell surface marker of CD3⁺CD8⁺CD197⁻CD45RO⁺CD28⁺CD95⁺ indicates a transitional memory cytotoxic T cell.

The present invention further provides an immunophenotyping kit for identifying a subset of T lymphocyte, comprising the following antibodies labeled by different fluorescent labels:
an anti-CD45 antibody, an anti-CD3 antibody, an anti-CD4 antibody, an anti-CD8 antibody, an anti-CD25 antibody, an anti-CD127 antibody, an anti-CD45RO antibody, an anti-CD197 antibody, an anti-CD28 antibody, and an anti-CD95 antibody.

In the immunophenotyping kit for identifying a subset of T lymphocyte provided by the present invention, the antibody labeled by a fluorescent label can be obtained by either coupling a fluorescent label with an antibody which are placed separately for use, or directly labeling an antibody by a fluorescent label for direct use.

In some embodiments of the present invention, the immunophenotyping kit for identifying a subset of T lymphocyte provided by the present invention comprises a fluorescent label and an antibody.

The antibodies are selected from the group consisting of:
an anti-CD45 antibody, an anti-CD3 antibody, an anti-CD4 antibody, an anti-CD8 antibody, an anti-CD25 antibody, an anti-CD127 antibody, an anti-CD45RO antibody, an anti-CD197 antibody, an anti-CD28 antibody, and an anti-CD95 antibody.

The fluorescent label includes, but is not limited to, FITC, APC-Cy7, Percp-cy5.5, BV510, BV421, Alexa Fluor 700, BV785, PE, BV605 and APC, and those skilled in the art can select according to the actual situation and need.

In some embodiments of the present invention, the anti-CD45 antibody is labeled by FITC as the fluorescent label in the immunophenotyping kit for identifying a subset of T lymphocyte provided by the present invention.

In some embodiments of the present invention, the anti-CD3 antibody is labeled by APC-Cy7 as the fluorescent label in the immunophenotyping kit for identifying a subset of T lymphocyte provided by the present invention.

In some embodiments of the present invention, the anti-CD4 antibody is labeled by Percp-Cy5.5 or BV650 as the fluorescent label in the immunophenotyping kit for identifying a subset of T lymphocyte provided by the present invention.

In some embodiments of the present invention, the anti-CD8 antibody is labeled by BV510 as the fluorescent label in the immunophenotyping kit for identifying a subset p of T lymphocyte provided by the present invention.

In some embodiments of the present invention, the anti-CD25 antibody is labeled by BV421 as the fluorescent label in the immunophenotyping kit for identifying a subset of T lymphocyte provided by the present invention.

In some embodiments of the present invention, the anti-CD127 antibody is labeled by Alexa Fluor 700 as the fluorescent label in the immunophenotyping kit for identifying a subset of T lymphocyte provided by the present invention.

In some embodiments of the present invention, the anti-CD45RO antibody is labeled by BV785 as the fluorescent label in the immunophenotyping kit for identifying a subset of T lymphocyte provided by the present invention.

In some embodiments of the present invention, the anti-CD197 antibody is labeled by PE as the fluorescent label in the immunophenotyping kit for identifying a subset of T lymphocyte provided by the present invention.

In some embodiments of the present invention, the anti-CD28 antibody is labeled by BV605 as the fluorescent label in the immunophenotyping kit for identifying a subset of T lymphocyte provided by the present invention.

In some embodiments of the present invention, the anti-CD95 antibody is labeled by APC as the fluorescent label in the immunophenotyping kit for identifying a subset of T lymphocyte provided by the present invention.

In an embodiment of the present invention, a fluorescent minus one (FMO) control is used as a control group in the immunophenotyping method for identifying a development subset of T lymphocyte provided by the present invention. In the detection of anti-CD25 antibody labeled by BV421 (BV421-CD25), the FMO control is set with the addition of all fluorescent antibodies except BV421-CD25. In the detection of anti-CD28 antibody labeled by BV605 (BV605-CD28), the FMO control is set with the addition of all fluorescent antibodies except BV605-CD28.

In the kit provided by the present invention, the fluorescent labels for fluorescently labeling each antibody are not limited by the present invention, and those skilled in the art can select a suitable fluorescent label, and a corresponding control, according to the actual situation.

In the present invention, in the detection by flow cytometry, the accuracy of the identification of target cells can be evaluated through the identification of the target cell subsets and the fluorescence intensity of the target cells. Clear identification of the target cell subset and high fluorescence intensity of the target cells indicate a high accuracy.

The present invention provides an immunophenotyping method and kit for identifying a subset of T lymphocyte. The immunophenotyping method for identifying a subset of T lymphocyte provided by present invention comprises mixing antibodies labeled by different fluorescent labels with a sample, incubating, performing detection by flow cytometry, collecting detection data, and analyzing the detection data. The antibodies are selected from the group consisting of an anti-CD45 antibody, an anti-CD3 antibody, an anti-CD4 antibody, an anti-CD8 antibody, an anti-CD25 antibody, an anti-CD127 antibody, an anti-CD45RO antibody, an anti-CD197 antibody, an anti-CD28 antibody, and an anti-CD95 antibody. The experimental results confirm that the present invention achieves a more comprehensive immunophenotyping and quantitative analysis for development subsets of T lymphocytes by flow cytometry using an optimal combination of antibodies against cell surface markers. In some embodiments of the present invention, the present invention provides a method that requires a small amount of sample, has simple operation and obvious results of identification of subset, and takes a short time. In some embodiments of the present invention, the method provided by the present invention has good reproducibility, obvious results of identification of subset and high accuracy, and can be widely used in immunophenotyping and quantitative analysis for a development subset of T lymphocyte.

### BRIEF DESCRIPTION OF DRAWINGS

FIG. 1 shows the results of classification of T lymphocytes in Example 1.

FIG. 2 shows the immunophenotyping results of development subset of T lymphocytes in Example 1; wherein, FIGS. 2-A and 2-B show the immunophenotyping result of Treg cells; FIGS. 2-D, 2-E, and 2-F show the immunophenotyping results of helper T cells; and FIGS. 2-H, 2-I, and 2-J show the immunophenotyping results of cytotoxic T cells.

FIG. 3 shows the results of classification of T lymphocytes in Example 2.

FIG. 4 shows the immunophenotyping results of development subset of T lymphocytes in Example 2; wherein, FIGS. 4-A and 2-B show the immunophenotyping result of Treg cells; FIGS. 4-D, 4-E, and 4-F show the immunophenotyping results of helper T cells; and FIGS. 4-H, 4-I, and 4-J show the immunophenotyping results of cytotoxic T cells.

FIG. 5 shows the results of classification of T lymphocytes in Example 3.

FIG. 6 shows the immunophenotyping results of development subsets of T lymphocytes in Example 3; wherein, FIGS. 6-A and 6-B show the immunophenotyping result of Treg cells; FIGS. 6-D, 6-E, and 6-F show the immunophenotyping results of helper T cells; and FIGS. 6-H, 6-I, and 6-J show the immunophenotyping results of cytotoxic T cells.

FIG. 7 shows the results of classification of T lymphocytes in Example 4.

FIG. 8 shows the immunophenotyping results of development subsets of T lymphocytes in Example 4; wherein, FIGS. 8-A and 8-B show the immunophenotyping result of Treg cells; FIGS. 8-D, 8-E, and 8-F show the immunophenotyping results of helper T cells; and FIGS. 8-H, 8-I, and 8-J show the immunophenotyping results of cytotoxic T cells.

FIG. 9 shows the results of classification of T lymphocytes in Example 5.

FIG. 10 shows the immunophenotyping results of development subsets of T lymphocytes in Example 5; wherein, FIGS. 10-A and 10-B show the immunophenotyping result of Treg cells; FIGS. 10-D, 10-E, and 10-F show the immunophenotyping results of helper T cells; and FIGS. 10-H, 10-I, and 10-J show the immunophenotyping results of cytotoxic T cells.

### DETAILED DESCRIPTION

The present invention provides an immunophenotyping method for identifying a subset of T lymphocyte and a kit. Those skilled in the art can learn from the content herein to appropriately improve the process parameters to realize the present invention. It should be particularly indicated that, all similar replacements and changes are obvious for those skilled in the art, which are deemed to be included in the present invention. The method and use of the present invention have been described through preferred embodiments, and those skilled apparently can make modifications or appropriate changes and combinations of the method and use herein without departing from the content, spirit and scope of the present invention to realize and apply the technology of the present invention.

The reagents and raw materials used in the immunophenotyping method and kit for identifying a development subset of T lymphocyte provided by the present invention are commercially available.

The fluorescent labels FITC, APC-Cy7, Percp-cy5.5, BV510, BV421, Alexa Fluor 700, BV785, PE, BV605, APC, and BV650 used in the present invention are common fluorescent labels that can be purchased in the market, and the antibody labeled by each fluorescent label can also be purchased in the market.

TABLE 1
Abbreviation

| Term | Abbreviation |
|---|---|
| Regulatory T cell | Treg |
| Naive T cell | TN |
| Stem-Cell Memory | TSCM |
| T cell | |
| Central Memory T | TCM |
| cell | |
| Effector Memory T | TEM |
| cell | |
| Transitional | TTM |
| Memory T cell | |

The present invention is further described below in conjunction with examples.

### Example 1 Immunophenotyping and Quantitative Analysis of Development Subsets of T Lymphocytes

### Experimental Material

The sample to be tested was an anticoagulant peripheral blood sample obtained from normal peripheral blood of a healthy volunteer (female, 36 years old).

An anti-CD45 antibody labeled by FITC, anti-CD3 antibody labeled by APC-Cy7, anti-CD4 antibody labeled by Percp-cy5.5, anti-CD8 antibody labeled by BV510, anti-CD25 antibody labeled by BV421, anti-CD127 antibody labeled by Alexa Flour 700, anti-CD45RO antibody labeled by BV785, anti-CD197 antibody labeled by PE, anti-CD28 antibody labeled by BV605 and anti-CD95 antibody labeled by APC were purchased from Biolegend.

A red blood cell lysis solution (cat RT122-02) was purchased from TIANGEN BIOTECH CO., LTD.

### Experimental Method

200 μL of sample to be tested was taken from 300 μL of an anticoagulant peripheral blood sample, and subjected to a haemocytometer to detect the absolute number of lymphocytes, which was detected to be 2.58×10⁹ cells/μL.

The remaining anticoagulant peripheral blood sample (100 μL) was used to detect development subsets of T lymphocytes, and to perform immunophenotyping and quantitative analysis of development subsets of T lymphocytes.

### Immunophenotyping of Development Subsets of T Lymphocytes

1. Two flow tubes were labeled as T-1 and T-2, wherein T-1 was the control group and T-2 was the detection group. The two flow tubes T-1 and T-2 were added with 50 μL of the sample to be tested respectively, and then added with 1 mL of red blood cell lysis solution respectively to obtain two mixtures. The obtained mixtures were fully mixed by vortex, then placed in a water bath at 37° C. for 6-8 minutes, and centrifuged at 500 g for 5 minutes. Then the supernatant was discarded.
2. The following antibodies were added to T-1 and T-2 flow tubes according to Table 2 to obtain two mixtures, which were fully mixed by vortex and incubated at room temperature in dark for 20 minutes.

TABLE 2
Volume of antibody added in each flow tube

| Flow tube | T-1 (FMO control) | T-2 |
|---|---|---|
| Antibody | 1 μL of anti-CD45 | 1 μL of anti-CD45 |
| labeled by | antibody labeled | antibody labeled |
| fluorescent | by FITC | by FITC |
| label | 1 μL of anti-CD3 | 1 μL of anti-CD3 |
| | antibody labeled | antibody labeled |
| | by APC-cy7 | by APC-cy7 |
| | 1 μL of anti-CD4 | 1 μL of anti-CD4 |
| | antibody labeled | antibody labeled |
| | by Percp-cy5.5 | by Percp-cy5.5 |
| | 1 μL of anti-CD8 | 1 μL of anti-CD8 |
| | antibody labeled | antibody labeled |
| | by BV510 | by BV510 |
| | | 1 μL of anti-CD25 |
| | | antibody labeled |
| | | by BV421 |
| | 1 μL of anti-CD127 | 1 μL of anti-CD127 |
| | antibody labeled | antibody labeled |
| | by Alexa Flour 700 | by Alexa Flour 700 |
| | 1 μL of anti-CD45RO | 1 μL of anti-CD45RO |
| | antibody labeled | antibody labeled |
| | by BV785 | by BV785 |
| | 1 μL of anti-CD197 | 1 μL of anti-CD197 |
| | antibody labeled | antibody labeled |
| | by PE | by PE |
| | | 1 μL of anti-CD28 |
| | | antibody labeled |
| | | by BV605 |
| | 1 μL of anti-CD95 | 1 μL of anti-CD95 |
| | antibody labeled | antibody labeled |
| | by APC | by APC |

3. 1 mL of PBS was added to each flow tube to obtain a mixture. The mixture was centrifuged at 500 g for 5 minutes, washed once and then added with 200 μL of PBS for suspension. The detection was performed on a flow cytometer and the results were analyzed.

### Result Analysis

According to the above experiment, the relative number (percentage) of each development subset of T lymphocytes and the absolute number of lymphocytes were collected. The absolute number of each development subset of T-lymphocytes was calculated as the relative number (percentage) of each development subset of T-lymphocytes×the absolute number of T lymphocytes.

### Results of Immunophenotyping and Quantitative Analysis of T Lymphocytes Using FlowJo Analysis Software

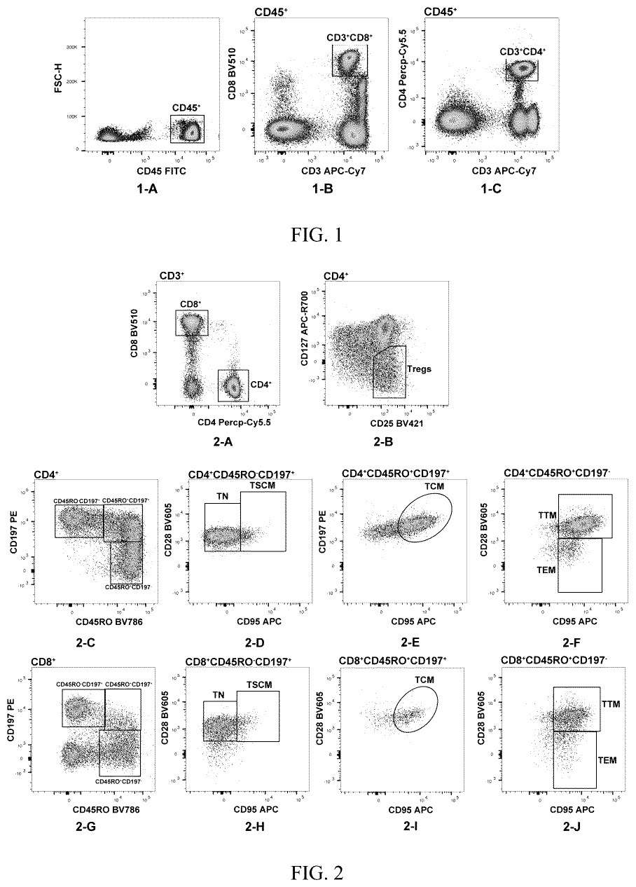

The immunophenotyping results of T lymphocytes are shown in FIG. 1, from which the relative numbers (percentages) of T cells, Tc cells, Th cells, and Treg cells were obtained. The absolute number of each T cell subset was calculated according to the absolute number of lymphocytes and the relative number (percentage) of each T cell subset. The specific experimental results are shown in Table 3.

TABLE 3
Cell surface markers for the identification of each T cell subset, and
the relative number and the absolute number of each T cell subset

| | | The relative number | |
|---|---|---|---|
| | | (percentage relative to the | |
| | | total number of | The absolute |
| T cell | Cell surface | lymphocytes) of T cell | number of T cell |
| subset | marker | subset | subset |
| T cell | CD3⁺ | 53.4% | 1377 cells/μL |
| Tc cell | CD3⁺CD8⁺ | 25.6% | 353 cells/μL |
| Th cell | CD3⁺CD4⁺ | 44.7% | 615 cells/μL |
| | CD4⁺/CD8⁺ | 1.75 | |
| | Ratio | | |

### Results of Immunophenotyping and Quantitative Analysis of Development Subset of T Lymphocytes Using FlowJo Analysis Software

The immunophenotyping results of development subsets of T lymphocytes are shown in FIGS. 2-A, 2-B, 2-C, 2-D, 2-E, 2-F, 2-G, 2-H, 2-I and 2-J. It can be seen that the relative number (percentage) of various development subsets of T lymphocytes is the percentage relative to the number of T cells. The absolute number of the development subsets of T lymphocytes was calculated according to the absolute number of T lymphocytes and the relative number (percentage) of each cell subset.

### 1. Regulatory T Cells (Treg Cells)

In FIG. 2-B, CD3⁺CD4⁺CD25⁺CD127⁻ indicated the target cell population. It can be detected by the flow cytometer that the target cells accounted for 18.86% of CD4⁺ T cells. The absolute number of Treg cells was calculated to be 116 cells/μL according to the absolute number of lymphocytes detected by the haemocytometer.

### 1. Helper T-Cells Subsets

(1) It can be seen from FIG. 2-D that the naive helper T cells (i.e., CD4⁺Naive, CD3⁺CD4⁺CD197⁺CD45RO⁻CD28⁺CD95⁻) had a relative number of 26.67% (percentage of CD4⁺ T cells) and an absolute number of 164 cells/μL; and the stem cell-like memory helper T cells (i.e., TSCM, CD3⁺CD4⁺CD197⁺CD45RO⁻CD28⁺CD95+) had a relative number of 2.62% and an absolute number of 16 cells/μL. (2) It can be seen from FIG. 2-E that the central memory helper T cells (i.e., TCM, CD3⁺CD4⁺CD197⁺CD45RO⁺CD28⁺CD95⁺) had a relative number of 23.67% and an absolute number of 146 cells/μL. (3) It can be seen from FIG. 2-F that the effector memory helper T cells (i.e., TEM, CD3⁺CD4⁺CD197⁻CD45RO⁺CD28⁻CD95⁺) had a relative number of 3.16% and an absolute number of 20 cells/μL, and the transitional memory helper T cells (i.e., TTM, CD3⁺CD4⁺CD197⁻CD45RO⁺CD28⁺CD95⁺) had a relative number of 30.93% and an absolute number of 190 cells/μL.

### 2. Cytotoxic T Cells

(1) It can be seen from FIG. 2-H that the naive cytotoxic T cells (i.e., CD8⁺Naive, CD3⁺CD8⁺CD197⁺CD45RO⁻CD28⁺CD95⁻) had a relative number of 26.39% (percentage of CD8⁺ T cells) and an absolute number of 93 cells/μL; and the stem cell-like memory cytotoxic T cells (i.e., TSCM, CD3⁺CD8⁺CD197⁺CD45RO⁻CD28⁺CD95⁺) had a relative number of 3.15% and an absolute number of 11 cells/μL.
(2) It can be seen from FIG. 2-I that the central memory cytotoxic T cells (i.e., TCM, CD3⁺CD8⁺CD197⁺CD45RO⁺CD28⁺CD95⁺) had a relative number of 4.25% and an absolute number of 15 cells/μL.
(3) It can be seen from FIG. 2-J that the effector memory cytotoxic T cells (i.e., TEM, CD3⁺CD8⁺CD197⁻CD45RO⁺CD28⁻CD95⁺) had a relative number of 4.15% and an absolute number of 15 cells/μL, and the transitional memory cytotoxic T cells (i.e., TTM, CD3⁺CD8⁺CD197⁻CD45RO⁺CD28⁺CD95⁺) had a relative number of 22.55% and an absolute number of 80 cells/μL.

TABLE 4
Relative and absolute numbers of each development subset
of T lymphocytes in normal humans

| | | The relative | The absolute number |
|---|---|---|---|
| | | number of T | of T cell subset |
| Cell | T cell subset | cell subset (%)* | (cell/μL)* |
| T cells | CD3⁺ | 69.5 (53.7-82.8) | 1342 (270-2586) |
| | | (percentage of | (absolute number in |
| | | lymphocytes ) | lymphocytes ) |
| Tc cells | CD3⁺CD8⁺ | 32.5 (14.8-48.4) | 438 (61-1118) |
| | | (percentage of T | (absolute number in |
| | | cells) | T cells) |
| Th cells | CD3⁺CD4⁺ | 61 (46.2-78) | 799 (199-1414) |
| | | (percentage of T | (absolute number in |
| | | cells) | T cells) |
| Regulatory | CD3⁺CD4⁺CD25⁺ | 7.9 (5.1-12.7) | 66 (28-142) |
| T cell | CD127- | (percentage of | (absolute number in |
| | | CD4⁺T cells) | CD4⁺T cells) |
| Helper | Naive helper T | 36.1 (7.2-68.9) | 289 (45-1079) |
| T cells | cells | (percentage of | (absolute number in |
| | | CD4⁺T cells) | CD4⁺T cells) |
| | Central memory | 39.5 (15-64.3) | 317 (70-671) |
| | helper T cell | (percentage of | (absolute number in |
| | | CD4⁺T cells) | CD4⁺T cells) |
| | Effector memory | 13.9 (4.9-43.6) | 119 (23-301) |
| | helper T cell | (percentage of | (absolute number in |
| | | CD4⁺T cells) | CD4⁺T cells) |
| Cytotoxic T | Naive cytotoxic T | 26.2 (2.6-72.4) | 111 (18-355) |
| cells | cell | (percentage of | (absolute number in |
| | | CD8⁺T cells) | CD8⁺T cells) |
| | Central memory | 11.7 (2.7-36.2) | 48 (7-206) |
| | cytotoxic T cell | (percentage of | (absolute number in |
| | | CD8⁺T cells) | CD8⁺T cells) |
| | Effector memory | 34.1 (4.7-60.1) | 155 (13-457) |
| | cytotoxic T cell | (percentage of | (absolute number in |
| | | CD8⁺T cells) | CD8⁺T cells) |

Wherein, “*” represents the experimental results reported in the prior art.

References: Anders H. Kverneland et al. Age and Gender Leucocytes Variances and References Values Generated Using the Standardized ONE-Study Protocol. 2016, Cytometry PartA, 89A:543-564.

TABLE 5
Relative and absolute numbers of each cell subset of T lymphocytes in normal children

| | | 1-6 months old | | 6-12 months old | | 12-18 years old | |
|---|---|---|---|---|---|---|---|
| | | | The absolute | | The absolute | | The absolute |
| | | The relative | number of | The relative | number of | The relative | number of |
| | | number of | T cell subset | number of | T cell subset | number of | T cell subset |
| | | T cell subset | (cell/μL)* | T cell subset | (cell/μL)* | T cell subset | (cell/μL)* |
| | | (%)* | (absolute | (%)* | (absolute | (%)* | (absolute |
| | T cell | (percentage | number in | (percentage | number in | (percentage | number in |
| Cell | subset | of T cells) | T cells) | of T cells) | T cells) | of T cells) | T cells) |
| T cells | CD3⁺ | 63.31 | 3488 | 64.04 | 3595 | 66.94 | 1661 |
| | | (54.28-71.67) | (2179-4424) | (55.32-73.11) | (2187-6352) | (56.84-75.02) | (1184-2144) |
| Tc cells | CD3⁺CD8⁺ | 18.45 | 979 | 22.98 | 1396 | 28.58 | 713 |
| | | (14.08-24.70) | (556-1687) | (15.88-31.48) | (686-2278) | (21.91-36.80) | (489-1009) |
| Th cells | CD3⁺CD4⁺ | 41.96 | 2279 | 38.38 | 2188 | 30.80 | 776 |
| | | (33.72-52.43) | (1461-3018) | (28.17-47.74) | (1125-3768) | (22.25-39.00) | (522-1084) |
| Regulatory | CD3⁺CD4⁺ | Treg (%): 0.8 (0.3-3.7) | | | | 1.5 | — |
| T cell | CD25⁺CD127⁻ | (percentage of lymphocytes) | | | | (0.8-4.3) | |
| | | Treg (cell/μL): 20(0-100) | | | | (percentage of | |
| | | (absolute number in lymphocytes) | | | | lymphocytes) | |
| Helper | Naive helper | 82.40 | 1839 | 78.45 | 1802 | 53.20 | 410 |
| T cells | T cells | (69.15-88.10) | (1170-2595) | (59.28-88.09) | (764-2972) | (39.50-66.26) | (230-627) |
| | Central memory | 15.10 | 318 | 18.40 | 362 | 37.80 | 257 |
| | helper T cell | (10.11-28.20) | (213-647) | (10.15-33.38) | (206-796) | (25.34-49.90) | (182-403) |
| | Effector memory | 1.02 | 21 (5-48) | 1.33 | 29 (11-60) | 8.10 | 60 |
| | helper T cell | (0.28-2.10) | | (0.42-3.96) | | (4.68-15.70 | (29-117) |
| Cytotoxic | Naive cytotoxic | 81.40 | 800 | 72.50 | 909 | 52.80 | 375 |
| T cells | T cell | (68.90-94.60) | (503-1276) | (47.36-92.45) | (535-1677) | (35.34-72.32) | (231-568) |
| | Central memory | 13.05 | 124 | 11.00 | 165 | 21.00 | 152 |
| | cytotoxic T cell | (5.14-25.55) | (41-305) | (4.82-24.11) | (51-316) | (10.96-31.00) | (74-228) |
| | Effector memory | 0.78 | 6 (1-70) | 2.01 | 28 (2-120) | 7.50 | 55 |
| | cytotoxic T cell | (0.10-4.95) | | (0.20-8.94) | | (2.38-15.84) | (16-109) |

Wherein, “*” represents the experimental results reported in the prior art. References: Yuan Ding et al. Reference values for peripheral blood lymphocyte subsets of healthy children in China. 2018, J ALLERGY CLIN IMMUNOL., 1-4; Marina Garcia-Prat et al. Extended Immunophenotyping Reference Values in a Healthy Pediatric Population. 2018, Cytometry Part B (Clinical Cytometry), DOI: 10.1002/cyto.b.21728.
“—”represents that the absolute number of Tregs in T cells was not found from the literature.

According to FIG. 1, it can be seen that the method used in this example could successfully achieve classification of T lymphocytes into CD3⁺ T cells, CD3⁺CD4⁺ cells and CD3⁺CD8⁺ cells, and could perform quantitative analysis to obtain the relative and absolute numbers of various T lymphocytes. According to Table 4, it can be seen that the relative and absolute numbers of the various cell subsets in the sample were within the reference range of normal humans, indicating that the experimental results of the present invention are stable and accurate.

According to FIG. 2-B, FIG. 2-D, FIG. 2-E, FIG. 2-F, FIG. 2-H, FIG. 2-I, and FIG. 2-J, it can be seen that the method used in this example could achieve accurate classification of T lymphocytes into Treg cells, naive helper T cells, stem cell-like memory helper T cells, central memory helper T cells, effector memory helper T cells, transitional memory helper T cells, naive cytotoxic T cells, stem cell-like memory cytotoxic T cells, central memory cytotoxic T cells, effector memory cytotoxic T cells, and transitional memory cytotoxic T cells, and could perform quantitative analysis to obtain the relative and absolute numbers of various development subsets of T lymphocytes. As can be seen from Table 3, the relative and absolute numbers of the various T cell subsets in the sample were within the reference range of normal human beings. Although the relative number of regulatory T cells increased, the absolute number thereof was within the reference range of normal human beings. The relative and absolute numbers of effector memory cytotoxic T cells were close to the reference values of normal human beings. It indicates that the experimental results of the present invention are accurate and stable.

In summary, the method provided by the present invention, with using a small amount of sample, achieves immunophenotyping of the development subsets of T lymphocytes and counts the content of each cell subset. The method could achieve accurate and clear immunophenotyping of development subsets of T lymphocytes.

### Example 2 Immunophenotyping and Quantitative Analysis of Development Subsets of T Lymphocytes

### Experimental Material

The sample to be tested was an anticoagulant peripheral blood sample obtained from a venous blood sample of a child (male, 14 years old) admitted to our hospital with neutrophil-mediated autoinflammation, who had signed informed consent.

An anti-CD45 antibody labeled by FITC, anti-CD3 antibody labeled by APC-Cy7, anti-CD4 antibody labeled by Percp-cy5.5, anti-CD8 antibody labeled by BV510, anti-CD25 antibody labeled by BV421, anti-CD127 antibody labeled by Alexa Flour 700, anti-CD45RO antibody labeled by BV785, anti-CD197 antibody labeled by PE, anti-CD28 antibody labeled by BV605 and anti-CD95 antibody labeled by APC were purchased from Biolegend.

A red blood cell lysis solution (cat RT122-02) was purchased from TIANGEN BIOTECH CO., LTD.

### Experimental Method

200 μL of sample to be tested was taken from 300 μL of an anticoagulant peripheral blood sample, and subjected to a haemocytometer to detect the absolute number of lymphocytes, which was detected to be 2.92×10⁹ cells/μL.

The remaining anticoagulant peripheral blood sample (100 μL) was used to detect development subsets of T lymphocytes, and to perform immunophenotyping and quantitative analysis of development subsets of T lymphocytes.

### Immunophenotyping of Development Subsets of T Lymphocytes

Same as the method for classification of T lymphocytes in Example 1, a flow tube as the detection group was added with 50 μL of the sample to be tested, and then added with 1 mL of red blood cell lysis solution to obtain a mixture. The mixture was fully mixed by vortex, then placed in a water bath at 37° C. for 6-8 minutes, and centrifuged at 500 g for 5 minutes. Then the supernatant was discarded.

An anti-CD45 antibody labeled by FITC, anti-CD3 antibody labeled by APC-Cy7, anti-CD4 antibody labeled by Percp-cy5.5, anti-CD8 antibody labeled by BV510, anti-CD25 antibody labeled by BV421, anti-CD127 antibody labeled by Alexa Flour 700, anti-CD45RO antibody labeled by BV785, anti-CD197 antibody labeled by PE, anti-CD28 antibody labeled by BV605 and anti-CD95 antibody labeled by APC, were added to the flow tube to obtain a mixture, which was then fully mixed by vortex and incubated at room temperature in dark for 20 minutes.

### Results of Immunophenotyping and Quantitative Analysis of T Lymphocytes Using FlowJo Analysis Software

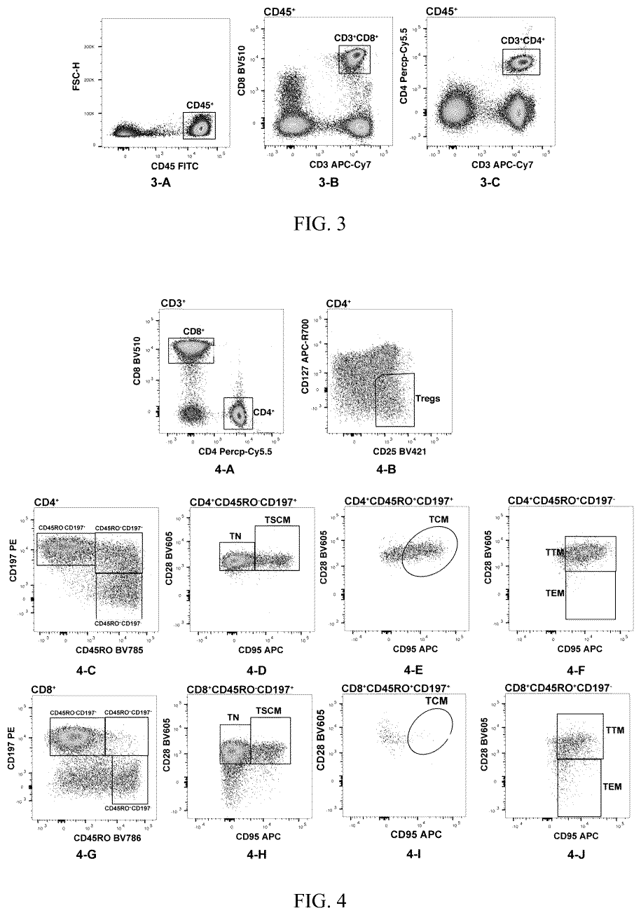

The immunophenotyping results of T lymphocytes are shown in FIG. 3, from which the relative numbers (percentages) of T cells, Tc cells, Th cells, and Treg cells were obtained. The absolute number of each T cell subset was calculated according to the absolute number of lymphocytes and the relative number (percentage) of each T cell subset. The specific experimental results are shown in Table 6.

TABLE 6
Cell surface markers for the identification of each T cell subset, and
the relative number and the absolute number of each T cell subset

| | | | The absolute |
|---|---|---|---|
| T cell | Cell surface | The relative number of T | number of T cell |
| subset | marker | cell subset (%) | subset (cell/μL) |
| T cell | CD3⁺ | 53.37% (percentage of the | 1558 cells/uL |
| | | total number of | (absolute number |
| | | lymphocytes) | in lymphocytes) |
| Tc cell | CD3⁺CD8⁺ | 50.2% (percentage of T | 782 cells/μL |
| | | cells) | (absolute number |
| | | | in T cells) |
| Th cell | CD3⁺CD4⁺ | 40.4% (percentage of T | 629 cells/μL |
| | | cells) | (absolute number |
| | | | in T cells) |
| | CD4⁺/CD8⁺ | 0.80 | |
| | Ratio | | |

### Results of Immunophenotyping and Quantitative Analysis of Development Subsets of T Lymphocytes Using FlowJo Analysis Software

The immunophenotyping results of development subsets of T lymphocytes are shown in FIGS. 4-A, 4-B, 4-C, 4-D, 4-E, 4-F, 4-G, 4-H, 4-I and 4-J. It can be seen that the relative number (percentage) of various development subsets of T lymphocytes was the percentage relative to the number of T cells. The absolute number of each development subset of T lymphocytes was calculated according to the absolute number of T lymphocytes and the relative number (percentage) of each development subset.

### 1. Regulatory T Cells (Treg Cells)

In FIG. 4-B, CD3⁺CD4⁺CD25⁺CD127⁻ indicated the target cell population. It can be detected by the flow cytometer that the target cells accounted for 3.48% of lymphocytes. The absolute number of Treg cells was calculated to be 102 cells/μL according to the absolute number of lymphocytes detected by the haemocytometer.

### 2. Helper T-Cells Subset

(1) It can be seen from FIG. 4-D that the naive helper T cells (i.e., CD4⁺Naive, CD3⁺CD4⁺CD197⁺CD45RO⁻CD28⁺CD95⁻) had a relative number of 16.90% (percentage of CD4⁺ T cells) and an absolute number of 264 cells/μL; and the stem cell-like memory helper T cells (i.e., TSCM, CD3⁺CD4⁺CD197⁺CD45RO⁻CD28⁺CD95⁺) had a relative number of 3.99% and an absolute number of 63 cells/μL.
(2) It can be seen from FIG. 4-E that the central memory helper T cells (i.e., TCM, CD3⁺CD4⁺CD197⁺CD45RO⁺CD28⁺CD95⁺) had a relative number of 5.75% and an absolute number of 90 cells/μL.
(3) It can be seen from FIG. 4-F that the effector memory helper T cells (i.e., TEM, CD3⁺CD4⁺CD197⁻CD45RO⁺CD28⁻CD95⁺) had a relative number of 0.10% and an absolute number of 2 cells/μL, and the transitional memory helper T cells (i.e., TTM, CD3⁺CD4⁺CD197⁻CD45RO⁺CD28⁺CD95⁺) had a relative number of 8.59% and an absolute number of 135 cells/μL.

### 3. Cytotoxic T Cells

(1) It can be seen from FIG. 4-H that the naive cytotoxic T cells (i.e., CD8⁺Naive, CD3⁺CD8⁺CD197⁺CD45RO⁻CD28⁺CD95⁻) had a relative number of 25.98% (percentage of CD8⁺ T cells) and an absolute number of 408 cells/μL; and the stem cell-like memory cytotoxic T cells (i.e., TSCM, CD3⁺CD8⁺CD197⁺CD45RO⁻CD28⁺CD95⁺) had a relative number of 4.43% and an absolute number of 70 cells/μL.
(2) It can be seen from FIG. 4-I that the central memory cytotoxic T cells (i.e., TCM, CD3⁺CD8⁺CD197⁺CD45RO⁺CD28⁺CD95⁺) had a relative number of 0.10% and an absolute number of 2 cells/μL.
(3) It can be seen from FIG. 4-J that the effector memory cytotoxic T cells (i.e., TEM, CD3⁺CD8⁺CD197⁻CD45RO⁺CD28⁻CD95⁺) had a relative number of 0.86% and an absolute number of 13 cells/μL, and the transitional memory cytotoxic T cells (i.e., TTM, CD3⁺CD8⁺CD197⁻CD45RO⁺CD28⁺CD95⁺) had a relative number of 3.42% and an absolute number of 54 cells/μL.

According to FIG. 3, it can be seen that the method used in this example could successfully achieve classification of T lymphocytes into CD3⁺ T cells, CD3⁺CD4⁺ cells and CD3⁺CD8⁺ cells, and could perform quantitative analysis to obtain the relative and absolute numbers of various T lymphocytes.

According to FIG. 4-B, FIG. 4-D, FIG. 4-E, FIG. 4-F, FIG. 4-H, FIG. 4-I, and FIG. 4-J, it can be seen that the method used in this example could achieve accurate classification of T lymphocytes into Treg cells, naive helper T cells, stem cell-like memory helper T cells, central memory helper T cells, effector memory helper T cells, transitional memory helper T cells, naive cytotoxic T cells, stem cell-like memory cytotoxic T cells, central memory cytotoxic T cells, effector memory cytotoxic T cells, and transitional memory cytotoxic T cells, and could perform quantitative analysis to obtain the relative and absolute numbers of various development subsets of T lymphocytes. According to Table 4, it can be seen that the relative and absolute numbers of central memory helper T cells, effector memory helper T cells, central memory cytotoxic T cells, and effector memory cytotoxic T cells in the sample were all lower than normal values, indicating that the development of T cells of this child was abnormal.

In summary, the method provided by the present invention, with using a small amount of sample, achieves immunophenotyping of the development subsets of T lymphocytes and counts the content of each development subset. The method could achieve accurate and clear immunophenotyping of development subsets of T lymphocytes.

### Example 3 Immunophenotyping and Quantitative Analysis of Development Subsets of T Lymphocytes

### Experimental Material

The sample to be tested was an anticoagulant peripheral blood sample obtained from a venous blood sample of a child (male, 10 months old) admitted to our hospital with liver failure, who had signed informed consent.

An anti-CD45 antibody labeled by FITC, anti-CD3 antibody labeled by APC-Cy7, anti-CD4 antibody labeled by Percp-cy5.5, anti-CD8 antibody labeled by BV510, anti-CD25 antibody labeled by BV421, anti-CD127 antibody labeled by Alexa Flour 700, anti-CD45RO antibody labeled by BV785, anti-CD197 antibody labeled by PE, anti-CD28 antibody labeled by BV605 and anti-CD95 antibody labeled by APC were purchased from Biolegend.

A red blood cell lysis solution (cat RT122-02) was purchased from TIANGEN BIOTECH CO., LTD.

### Experimental Method

200 μL of sample to be tested was taken from 300 μL of an anticoagulant peripheral blood sample, and subjected to a haemocytometer to detect the absolute number of lymphocytes, which was detected to be 5.14×10⁹ cells/μL.

The remaining anticoagulant peripheral blood sample (100 μL) was used to detect development subsets of T lymphocytes, and to perform immunophenotyping and quantitative analysis of development subsets of T lymphocytes.

### Immunophenotyping of Development Subsets of T Lymphocytes

Same as the method for classification of T lymphocytes in Example 1, a flow tube as the detection group was added with 50 μL of the sample to be tested, and then added with 1 mL of red blood cell lysis solution to obtain a mixture. The mixture was fully mixed by vortex, then placed in a water bath at 37° C. for 6-8 minutes, and centrifuged at 500 g for 5 minutes. Then the supernatant was discarded.

An anti-CD45 antibody labeled by FITC, anti-CD3 antibody labeled by APC-Cy7, anti-CD4 antibody labeled by Percp-cy5.5, anti-CD8 antibody labeled by BV510, anti-CD25 antibody labeled by BV421, anti-CD127 antibody labeled by Alexa Flour 700, anti-CD45RO antibody labeled by BV785, anti-CD197 antibody labeled by PE, anti-CD28 antibody labeled by BV605 and anti-CD95 antibody labeled by APC, were added to the flow tube to obtain a mixture, which was then fully mixed by vortex and incubated at room temperature in dark for 20 minutes

### Results of Immunophenotyping and Quantitative Analysis of T Lymphocytes Using FlowJo Analysis Software

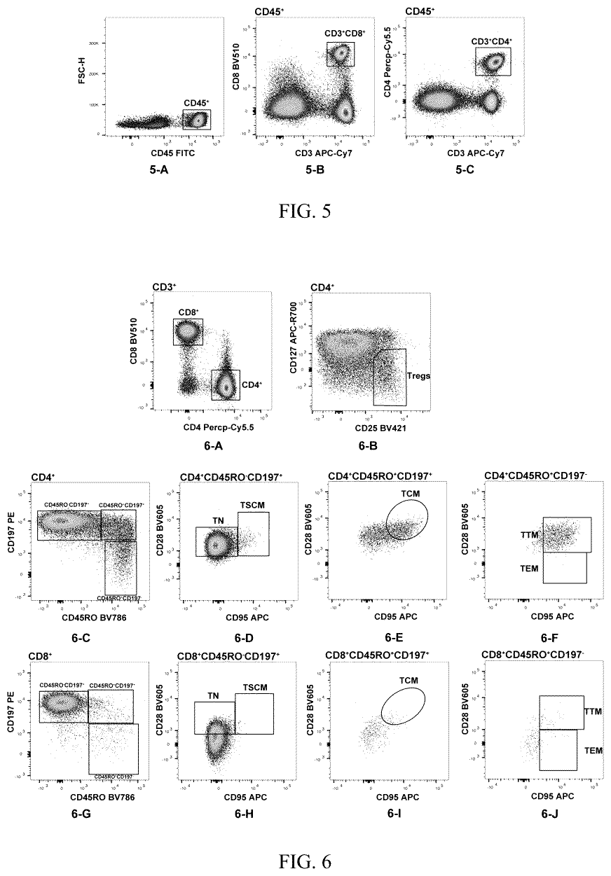

The immunophenotyping results of T lymphocytes are shown in FIG. 5, from which the relative numbers (percentages) of T cells, Tc cells, Th cells, and Treg cells were obtained. The absolute number of each T cell subset was calculated according to the absolute number of lymphocytes and the relative number (percentage) of each T cell subset. The specific experimental results are shown in Table 7.

TABLE 7
Cell surface markers for the identification of each T cell subset, and the
relative number and the absolute number of each T cell subset

| | | | The absolute |
|---|---|---|---|
| T cell | Cell surface | The relative number of T | number of T cell |
| subset | marker | cell subset (%) | subset (cell/μL) |
| T cell | CD3⁺ | 50.9% (percentage of the | 2616 cells/μL |
| | | total number of | (absolute number |
| | | lymphocytes) | in lymphocytes) |
| Tc cell | CD3⁺CD8⁺ | 34.5% (percentage of T | 902 cells/μL |
| | | cells) | (absolute number |
| | | | in T cells) |
| Th cell | CD3⁺CD4⁺ | 61.2% (percentage of T | 1600 cells/μL |
| | | cells) | (absolute number |
| | | | in T cells) |
| | CD4⁺/CD8⁺ | 1.77 | |
| | Ratio | | |

### Results of Immunophenotyping and Quantitative Analysis of Development Subsets of T Lymphocytes Using FlowJo Analysis Software

The immunophenotyping results of development subsets of T lymphocytes are shown in FIG. 6-A, 6-B, 6-C, 6-D, 6-E, 6-F, 6-G, 6-H, 6-I and 6-J. From FIG. 6, it can be seen that the relative number (percentage) of various development subsets of T lymphocytes is the percentage relative to the number of T cells. The absolute number of each development subset of T lymphocytes was calculated according to the absolute number of T lymphocytes and the relative number (percentage) of each cell subset.

### 1. Regulatory T Cells (Treg Cells)

In FIG. 6-1B, CD3⁺CD4⁺CD25⁺CD127⁻ indicated the target cell population. It can be detected by the flow cytometer that the target cells accounted for 2.04% of lymphocytes. The absolute number of Treg cells was calculated to be 105 cells/μL according to the absolute number of lymphocytes detected by the haemocytometer.

3. Helper T-cells subset
(1) It can be seen from FIG. 6-D that the naive helper T cells (i.e., CD4⁺Naive, CD3⁺CD4⁺CD197⁺CD45RO⁻CD28⁺CD95⁻) had a relative number of 49.64% and an absolute number of 1298 cells/μL; and the stem cell-like memory helper T cells (i.e., TSCM, CD3⁺CD4⁺CD197⁺CD45RO⁻CD28⁺CD95⁺) had a relative number of 0.44% and an absolute number of 11 cells/μL.
(2) It can be seen from FIG. 6-E that the central memory helper T cells (i.e., TCM, CD3⁺CD4⁺CD197⁺CD45RO⁺CD28⁺CD95⁺) had a relative number of 2.87% and an absolute number of 75 cells/μL.
(3) It can be seen from FIG. 6-F that the effector memory helper T cells (i.e., TEM, CD3⁺CD4⁺CD197⁻CD45RO⁺CD28⁻CD95⁺) had a relative number of 0.02% and an absolute number of 0.4 cells/μL, and the transitional memory helper T cells (i.e., TTM, CD3⁺CD4⁺CD197⁻CD45RO⁺CD28⁺CD95⁺) had a relative number of 2.09% and an absolute number of 55 cells/μL.

### 1. Cytotoxic T Cells

(1) It can be seen from FIG. 6-H that the naive cytotoxic T cells (i.e., CD8⁺Naive, CD3⁺CD8⁺CD197⁺CD45RO⁻CD28⁺CD95⁻) had a relative number of 10.47% (percentage of CD8⁺ T cells) and an absolute number of 274 cells/μL; and the stem cell-like memory cytotoxic T cells (i.e., TSCM, CD3⁺CD8⁺CD197⁺CD45RO⁻CD28⁺CD95⁺) had a relative number of 0.08% and an absolute number of 2 cells/μL.
(2) It can be seen from FIG. 6-I that the central memory cytotoxic T cells (i.e., TCM, CD3⁺CD8⁺CD197⁺CD45RO⁺CD28⁺CD95⁺) had a relative number of 0.11% and an absolute number of 3 cells/μL.
(3) It can be seen from FIG. 6-J that the effector memory cytotoxic T cells (i.e., TEM, CD3⁺CD8⁺CD197⁻CD45RO⁺CD28⁻CD95⁺) had a relative number of 0.01% and an absolute number of 0.2 cell/μL, and the transitional memory cytotoxic T cells (i.e., TTM, CD3⁺CD8⁺CD197⁻CD45RO⁺CD28⁺CD95⁺) had a relative number of 0.08% and an absolute number of 2 cells/μL.

According to FIG. 5, it can be seen that the method used in this example could successfully achieve classification of T lymphocytes into CD3⁺ T cells, CD3⁺CD4⁺ cells and CD3⁺CD8⁺ cells, and could perform quantitative analysis to obtain the relative and absolute numbers of various T lymphocytes.

According to FIG. 6-B, FIG. 6-D, FIG. 6-E, FIG. 6-F, FIG. 6-H, FIG. 6-I, and FIG. 6-J, it can be seen that the method used in this example could achieve accurate classification of T lymphocytes into Treg cells, naive helper T cells, stem cell-like memory helper T cells, central memory helper T cells, effector memory helper T cells, transitional memory helper T cells, naive cytotoxic T cells, stem cell-like memory cytotoxic T cells, central memory cytotoxic T cells, effector memory cytotoxic T cells, and transitional memory cytotoxic T cells, and could perform quantitative analysis to obtain the relative and absolute numbers of various development subsets of T lymphocytes. According to Table 4, it can be seen that the relative and absolute numbers of central memory helper T cells, effector memory helper T cells, naive cytotoxic T cells, central memory cytotoxic T cells, and effector memory cytotoxic T cells in the sample were all lower than normal values, indicating that the development of T cells of this child was abnormal.

In summary, the method provided by the present invention, with using a small amount of sample, achieves immunophenotyping of the development subsets of T lymphocytes and counts the content of each development subset. The method could achieve accurate and clear immunophenotyping of development subsets of T lymphocytes.

### Example 4 Immunophenotyping and Quantitative Analysis of Development Subsets of T Lymphocytes

### Experimental Material

The sample to be tested was an anticoagulant peripheral blood sample obtained from a venous blood sample of a child (male, 4 months old) admitted to our hospital with biliary atresia, who had signed informed consent.

An anti-CD45 antibody labeled by FITC, anti-CD3 antibody labeled by APC-Cy7, anti-CD4 antibody labeled by Percp-cy5.5, anti-CD8 antibody labeled by BV510, anti-CD25 antibody labeled by BV421, anti-CD127 antibody labeled by Alexa Flour 700, anti-CD45RO antibody labeled by BV785, anti-CD197 antibody labeled by PE, anti-CD28 antibody labeled by BV605 and anti-CD95 antibody labeled by APC were purchased from Biolegend.

A red blood cell lysis solution (cat RT122-02) was purchased from TIANGEN BIOTECH CO., LTD.

### Experimental Method:

200 μL of sample to be tested was taken from 300 μL of an anticoagulant peripheral blood sample, and subjected to a haemocytometer to detect the absolute number of lymphocytes, which was detected to be 7.31×10⁹ cells/μL.

The remaining anticoagulant peripheral blood sample (100 μL) was used to detect development subsets of T lymphocytes, and to perform immunophenotyping and quantitative analysis of development subsets of T lymphocytes.

### Immunophenotyping of Development Subsets of T Lymphocytes

Same as the method for classification of T lymphocytes in Example 4, a flow tube as the detection group was added with 50 μL of the sample to be tested, and then added with 1 mL of red blood cell lysis solution to obtain a mixture. The mixture was fully mixed by vortex, then placed in a water bath at 37° C. for 6-8 minutes, and centrifuged at 500 g for 5 minutes. Then the supernatant was discarded.

An anti-CD45 antibody labeled by FITC, anti-CD3 antibody labeled by APC-Cy7, anti-CD4 antibody labeled by Percp-cy5.5, anti-CD8 antibody labeled by BV510, anti-CD25 antibody labeled by BV421, anti-CD127 antibody labeled by Alexa Flour 700, anti-CD45RO antibody labeled by BV785, anti-CD197 antibody labeled by PE, anti-CD28 antibody labeled by BV605 and anti-CD95 antibody labeled by APC, were added to the flow tube to obtain a mixture, which was then fully mixed by vortex and incubated at room temperature in dark for 20 minutes.

### Results of Immunophenotyping and Quantitative Analysis of T Lymphocytes Using FlowJo Analysis Software

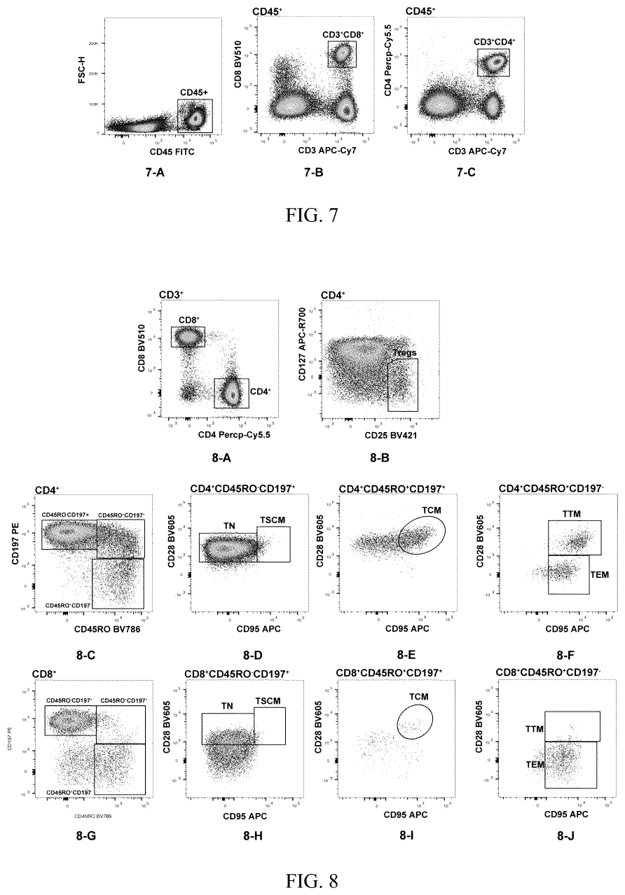

The immunophenotyping results of T lymphocytes are shown in FIG. 7, from which the relative numbers (percentages) of T cells, Tc cells, Th cells, and Treg cells were obtained. The absolute number of each T cell subset was calculated according to the absolute number of lymphocytes and the relative number (percentage) of each T cell subset. The specific experimental results are shown in Table 8.

TABLE 8
Cell surface markers for the identification of each T cell subset, and
the relative number and the absolute number of each T cell subset

| | | | The absolute |
|---|---|---|---|
| T cell | Cell surface | The relative number of T | number of T cell |
| subset | marker | cell subset (%) | subset (cell/μL) |
| T cell | CD3⁺ | 28.3% (percentage of the | 2069 cells/μL |
| | | total number of | (absolute number |
| | | lymphocytes) | in lymphocytes) |
| Tc cell | CD3⁺CD8⁺ | 25.7% (percentage of T | 532 cells/μL |
| | | cells) | (absolute number |
| | | | in T cells) |
| Th cell | CD3⁺CD4⁺ | 69% (percentage of T cells) | 1428 cells/μL |
| | | | (absolute number |
| | | | in T cells) |
| | CD4⁺/CD8⁺ | 2.68 | |
| | Ratio | | |

### Results of Immunophenotyping and Quantitative Analysis of Development Subsets of T Lymphocytes Using FlowJo Analysis Software

The immunophenotyping results of development subsets of T lymphocytes are shown in FIG. 8-A, 8-B, 8-C, 8-D, 8-E, 8-F, 8-G, 8-H, 8-I and 8-J. From FIG. 8, it can be seen that the relative number (percentage) of various development subsets of T lymphocytes is the percentage relative to the number of T cells. The absolute number of each development subset of T lymphocytes was calculated according to the absolute number of T lymphocytes and the relative number (percentage) of each cell subset.

### 1. Regulatory T Cells (Treg Cells)

In FIG. 8-B, CD3⁺CD4⁺CD25⁺CD127⁻ indicated the target cell population. It can be detected by the flow cytometer that the target cells accounted for 0.96% of lymphocytes. The absolute number of Treg cells was calculated to be 70 cells/μL according to the absolute number of lymphocytes detected by the haemocytometer.

### 4. Helper T-Cells Subset

(1) It can be seen from FIG. 8-D that the naive helper T cells (i.e., CD4⁺Naive, CD3⁺CD4⁺CD197⁺CD45RO⁻CD28⁺CD95⁻) had a relative number of 55.15% and an absolute number of 1141 cells/μL; and the stem cell-like memory helper T cells (i.e., TSCM, CD3⁺CD4⁺CD197⁺CD45RO⁻CD28⁺CD95⁺) had a relative number of 0.71% and an absolute number of 15 cells/μL.
(2) It can be seen from FIG. 8-E that the central memory helper T cells (i.e., TCM, CD3⁺CD4⁺CD197⁺CD45RO⁺CD28⁺CD95⁺) had a relative number of 3.65% and an absolute number of 75 cells/μL.
(3) It can be seen from FIG. 8-F that the effector memory helper T cells (i.e., TEM, CD3⁺CD4⁺CD197⁻CD45RO⁺CD28⁻CD95⁺) had a relative number of 1.46% and an absolute number of 30 cells/μL, and the transitional memory helper T cells (i.e., TTM, CD3⁺CD4⁺CD197⁻CD45RO⁺CD28⁺CD95⁺) had a relative number of 1.76% and an absolute number of 36 cells/μL.

### 1. Cytotoxic T Cells

(1) It can be seen from FIG. 8-H that the naive cytotoxic T cells (i.e., CD8⁺Naive, CD3⁺CD8⁺CD197⁺CD45RO⁻CD28⁺CD95⁻) had a relative number of 7.77% (percentage of CD8⁺ T cells) and an absolute number of 160 cells/μL; and the stem cell-like memory cytotoxic T cells (i.e., TSCM, CD3⁺CD8⁺CD197⁺CD45RO⁻CD28⁺CD95⁺) had a relative number of 0.16% and an absolute number of 3 cells/μL.
(2) It can be seen from FIG. 8-I that the central memory cytotoxic T cells (i.e., TCM, CD3⁺CD8⁺CD197⁺CD45RO⁺CD28⁺CD95⁺) had a relative number of 0.15% and an absolute number of 3 cells/μL.
(3) It can be seen from FIG. 8-J that the effector memory cytotoxic T cells (i.e., TEM, CD3⁺CD8⁺CD197⁻CD45RO⁺CD28⁻CD95⁺) had a relative number of 1.92% and an absolute number of 40 cells/μL, and the transitional memory cytotoxic T cells (i.e., TTM, CD3⁺CD8⁺CD197⁻CD45RO⁺CD28⁺CD95⁺) had a relative number of 0.14% and an absolute number of 3 cells/μL.

According to FIG. 7, it can be seen that the method used in this example could successfully achieve classification of T lymphocytes into CD3⁺ T cells, CD3⁺CD4⁺ cells and CD3⁺CD8⁺ cells, and could perform quantitative analysis to obtain the relative and absolute numbers of various T lymphocytes.

According to FIG. 8-B, FIG. 8-D, FIG. 8-E, FIG. 8-F, FIG. 8-H, FIG. 8-I, and FIG. 8-J, it can be seen that the method used in this example could achieve accurate classification of T lymphocytes into Treg cells, naive helper T cells, stem cell-like memory helper T cells, central memory helper T cells, effector memory helper T cells, transitional memory helper T cells, naive cytotoxic T cells, stem cell-like memory cytotoxic T cells, central memory cytotoxic T cells, effector memory cytotoxic T cells, and transitional memory cytotoxic T cells, and could perform quantitative analysis to obtain the relative and absolute numbers of various development subsets of T lymphocytes. According to Table 4, it can be seen that the relative and absolute numbers of central memory helper T cells, effector memory helper T cells, naive cytotoxic T cells, central memory cytotoxic T cells, and effector memory cytotoxic T cells in the sample were all lower than normal values, indicating that the development of T cells of this child was abnormal.

In summary, the method provided by the present invention, with using a small amount of sample, achieves immunophenotyping of the development subsets of T lymphocytes and counts the content of each development subset. The method could achieve accurate and clear immunophenotyping of development subsets of T lymphocytes.

### Example 5 Immunophenotyping and Quantitative Analysis of Development Subsets of T Lymphocytes

### Experimental Material

The sample to be tested was an anticoagulant peripheral blood sample obtained from normal peripheral blood of a healthy volunteer (female, 26 years old).

An anti-CD45 antibody labeled by FITC, anti-CD3 antibody labeled by APC-Cy7, anti-CD4 antibody labeled by Percp-cy5.5, anti-CD8 antibody labeled by BV510, anti-CD25 antibody labeled by BV421, anti-CD127 antibody labeled by Alexa Flour 700, anti-CD45RO antibody labeled by BV785, anti-CD197 antibody labeled by PE, anti-CD28 antibody labeled by BV605 and anti-CD95 antibody labeled by APC were purchased from Biolegend.

A lymphocyte separation solution was purchased from Dakewe Biotech Co., Ltd.

### Experimental Method

200 μL of sample to be tested was taken from 3 mL of an anticoagulant peripheral blood sample, and subjected to a haemocytometer to detect the absolute number of lymphocytes, which was detected to be 1.13×10⁹ cells/μL.

Peripheral blood mononuclear cells (PBMCs) were extracted from the remaining anticoagulant peripheral blood sample, which were then used to perform immunophenotyping analysis and quantitative analysis of development subsets of T lymphocytes.

### Immunophenotyping of Development Subsets of T Lymphocytes

The anticoagulant peripheral blood was diluted with PBS, added with a lymphocyte separation solution, and centrifuged at 800 g for 20 minutes. Then a white layer (PBMC layer) at the interface was collected into a flow tube.

An anti-CD45 antibody labeled by FITC, anti-CD3 antibody labeled by APC-Cy7, anti-CD4 antibody labeled by Percp-cy5.5, anti-CD8 antibody labeled by BV510, anti-CD25 antibody labeled by BV421, anti-CD127 antibody labeled by Alexa Flour 700, anti-CD45RO antibody labeled by BV785, anti-CD197 antibody labeled by PE, anti-CD28 antibody labeled by BV605 and anti-CD95 antibody labeled by APC, were added to the flow tube with 1×10⁶ cells to obtain a mixture, which was then fully mixed by vortex and incubated at room temperature in dark for 20 minutes.

### Results of Immunophenotyping and Quantitative Analysis of T Lymphocytes Using FlowJo Analysis Software

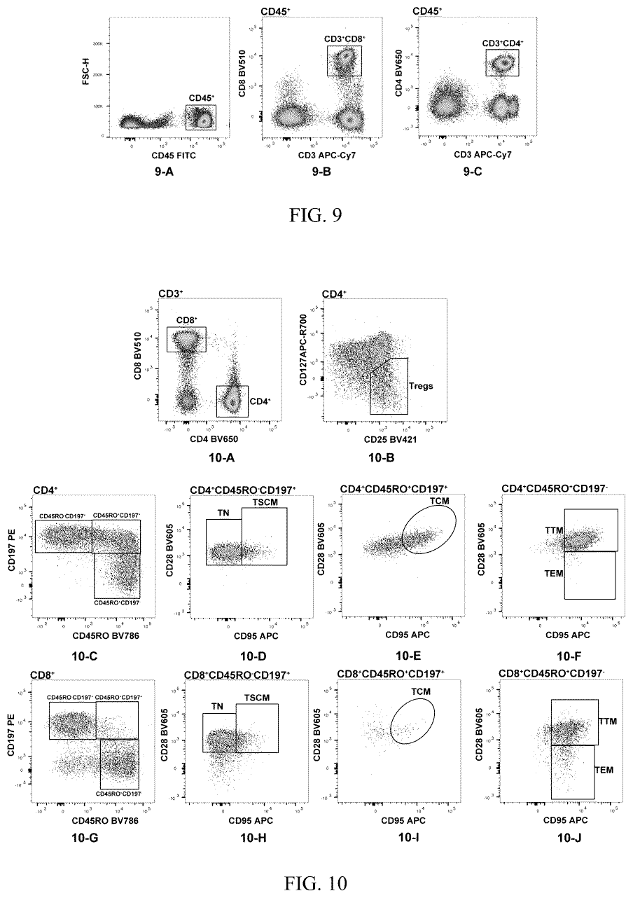

The immunophenotyping results of T lymphocytes are shown in FIG. 9, from which the relative numbers (percentages) of T cells, Tc cells, Th cells, and Treg cells were obtained. The absolute number of each T cell subset was calculated according to the absolute number of lymphocytes and the relative number (percentage) of each T cell subset. The specific experimental results are shown in Table 9.

TABLE 9
Cell surface markers for the identification of each T cell subset, and
the relative number and the absolute number of each T cell subset

| | | | The absolute |
|---|---|---|---|
| T cell | Cell surface | The relative number of T | number of T cell |
| subset | marker | cell subset (%) | subset (cell/μL) |
| T cell | CD3⁺ | 54.3% (percentage of the | 618 cells/μL |
| | | total number of | (absolute number |
| | | lymphocytes) | in lymphocytes) |
| Tc cell | CD3⁺CD8⁺ | 35% (percentage of T cells) | 216 cells/μL |
| | | | (absolute number |
| | | | in T cells) |
| Th cell | CD3⁺CD4⁺ | 48% (percentage of T cells) | 297 cells/μL |
| | | | (absolute number |
| | | | in T cells) |
| | CD4⁺/CD8⁺ | 1.37 | |
| | Ratio | | |

### Results of Immunophenotyping and Quantitative Analysis of Development Subsets of T Lymphocytes Using FlowJo Analysis Software

The immunophenotyping results of development subsets of T lymphocytes are shown in FIG. 10-A, 10-B, 10-C, 10-D, 10-E, 10-F, 10-G, 10-H, 10-I and 10-J. From FIG. 10, it can be seen that the relative number (percentage) of various development subsets of T lymphocytes is the percentage relative to the number of T cells. The absolute number of each development subset of T lymphocytes was calculated according to the absolute number of T lymphocytes and the relative number (percentage) of each cell subset.

### 1. Regulatory T Cells (Treg Cells)

In FIG. 9-B, CD3⁺CD4⁺CD25⁺CD127⁻ indicated the target cell population. It can be detected by the flow cytometer that the target cells accounted for 4.12% of lymphocytes. The absolute number of Treg cells was calculated to be 25 cells/μL according to the absolute number of lymphocytes detected by the haemocytometer.

### 5. Helper T-Cells Subset

(1) It can be seen from FIG. 10-D that the naive helper T cells (i.e., CD4⁺Naive, CD3⁺CD4⁺CD197⁺CD45RO⁻CD28⁺CD95⁻) had a relative number of 14.07% and an absolute number of 87 cells/μL; and the stem cell-like memory helper T cells (i.e., TSCM, CD3⁺CD4⁺CD197⁺CD45RO⁻CD28⁺CD95⁺) had a relative number of 2.47% and an absolute number of 15 cells/μL.
(2) It can be seen from FIG. 10-E that the central memory helper T cells (i.e., TCM, CD3⁺CD4⁺CD197⁺CD45RO⁺CD28⁺CD95⁺) had a relative number of 7.71% and an absolute number of 48 cells/μL.
(3) It can be seen from FIG. 10-F that the effector memory helper T cells (i.e., TEM, CD3⁺CD4⁺CD197⁻CD45RO⁺CD28⁻CD95⁺) had a relative number of 0.26% and an absolute number of 2 cells/μL, and the transitional memory helper T cells (i.e., TTM, CD3⁺CD4⁺CD197⁻CD45RO⁺CD28⁺CD95⁺) had a relative number of 14.87% and an absolute number of 92 cells/μL.

### 2. Cytotoxic T Cells

(1) It can be seen from FIG. 10-H that the naive cytotoxic T cells (i.e., CD8⁺Naive, CD3⁺CD8⁺CD197⁺CD45RO⁻CD28⁺CD95⁻) had a relative number of 14.23% (percentage of CD8⁺ T cells) and an absolute number of 88 cells/μL; and the stem cell-like memory cytotoxic T cells (i.e., TSCM, CD3⁺CD8⁺CD197⁺CD45RO⁻CD28⁺CD95⁺) had a relative number of 2.47% and an absolute number of 15 cells/μL.
(2) It can be seen from FIG. 10-I that the central memory cytotoxic T cells (i.e., TCM, CD3⁺CD8⁺CD197⁺CD45RO⁺CD28⁺CD95⁺) had a relative number of 0.35% and an absolute number of 2 cells/μL.
(3) It can be seen from FIG. 10-J that the effector memory cytotoxic T cells (i.e., TEM, CD3⁺CD8⁺CD197⁻CD45RO⁺CD28⁻CD95⁺) had a relative number of 1.52% and an absolute number of 9 cells/μL, and the transitional memory cytotoxic T cells (i.e., TTM, CD3⁺CD8⁺CD197⁻CD45RO⁺CD28⁺CD95⁺) had a relative number of 8.90% and an absolute number of 55 cells/μL.

According to FIG. 9, it can be seen that the method used in this example could successfully achieve classification of T lymphocytes into CD3⁺ T cells, CD3⁺CD4⁺ cells and CD3⁺CD8⁺ cells, and could perform quantitative analysis to obtain the relative and absolute numbers of various T lymphocytes.

According to FIG. 10-B, FIG. 10-D, FIG. 10-E, FIG. 10-F, FIG. 10-H, FIG. 10-I, and FIG. 10-J, it can be seen that the method used in this example could achieve accurate classification of T lymphocytes into Treg cells, naive helper T cells, stem cell-like memory helper T cells, central memory helper T cells, effector memory helper T cells, transitional memory helper T cells, naive cytotoxic T cells, stem cell-like memory cytotoxic T cells, central memory cytotoxic T cells, effector memory cytotoxic T cells, and transitional memory cytotoxic T cells, and could perform quantitative analysis to obtain the relative and absolute numbers of various development subsets of T lymphocytes. As can be seen from Table 9, it can be seen that the relative and absolute numbers of each T cell subset in the sample were all basically at or close to the reference values of normal human beings, indicating that the experimental results of the present invention are accurate and stable.

In summary, the method provided by the present invention, with using a small amount of sample, achieves immunophenotyping of the development subsets of T lymphocytes and counts the content of each development subset. The method could achieve accurate and clear immunophenotyping of development subsets of T lymphocytes.

The above are only preferred embodiments of the present invention. It should be noted that those skilled in the art can make several improvements and modifications without departing from the principle of the present invention. These improvements and modifications shall fall within the protection scope of the present invention.

1. An immunophenotyping method for identifying a subset of T lymphocyte, comprising mixing a composition 1 comprising antibodies labeled by different fluorescent labels with a sample, incubating, performing detection by flow cytometry, collecting detection data, and identifying the subset based on the detection data;
wherein the composition 1 consists of an anti-CD45 antibody, an anti-CD3 antibody, an anti-CD4 antibody, an anti-CD8 antibody, an anti-CD25 antibody, an anti-CD127 antibody, an anti-CD45RO antibody, an anti-CD28 antibody, an anti-CD197 antibody and an anti-CD95 antibody; and
wherein, the anti-CD45 antibody is labeled with a fluorescent label of FITC;
the anti-CD3 antibody is labeled with a fluorescent label of APC-Cy7:
the anti-CD4 antibody is labeled with a fluorescent label of Percp-cy5.5 or BV650;
the anti-CD8 antibody is labeled with a fluorescent label of BV510;
the anti-CD25 antibody is labeled with a fluorescent label of BV421;
the anti-CD127 antibody is labeled with a fluorescent label of Alexa Fluor 700;
the anti-CD45RO antibody is labeled with a fluorescent label of BV785;
the anti-CD197 antibody is labeled with a fluorescent label of PE:
the anti-CD28 antibody is labeled with a fluorescent label of BV605;
the anti-CD95 antibody is labeled with a fluorescent label of APC; and
a criterion of the identifying is:
a cell surface marker of CD3⁺CD4⁺CD25⁺CD127⁻ indicates a regulatory T cell,
a cell surface marker of CD3⁺CD4⁺CD197⁺CD45RO⁻CD28⁺CD95⁻ indicates a naive helper T cell,
a cell surface marker of CD3⁺CD4⁺CD197⁺CD45RO⁻CD28⁺CD95⁺ indicates a stem cell-like memory helper T cell,
a cell surface marker of CD3⁺CD4⁺CD197⁺CD45RO⁺CD28⁺CD95⁺ indicates a central memory helper T cell,
a cell surface marker of CD3⁺CD4⁺CD197⁻CD45RO⁺CD28⁻CD95⁺ indicates an effector memory helper T cell,
a cell surface marker of CD3⁺CD4⁺CD197⁻CD45RO⁺CD28⁺CD95⁺ indicates a transitional memory helper T cell,
a cell surface marker of CD3⁺CD8⁺CD197⁺CD45RO⁻CD28⁺CD95⁻ indicates a naive cytotoxic T cell,
a cell surface marker of CD3⁺CD8⁺CD197⁺CD45RO⁻CD28⁺CD95⁺ indicates a stem cell-like memory cytotoxic T cell,
a cell surface marker of CD3⁺CD8⁺CD197⁺CD45RO⁺CD28⁺CD95⁺ indicates a central memory cytotoxic T cell,
a cell surface marker of CD3⁺CD8⁺CD197⁻CD45RO⁺CD28⁻CD95⁺ indicates an effector memory cytotoxic T cell, and
a cell surface marker of CD3⁺CD8⁺CD197⁻CD45RO⁺CD28⁺CD95⁺ indicates a transitional memory cytotoxic T cell.

2. The immunophenotyping method according to claim 1, further comprising a step of counting the number of the T lymphocytes in the sample by:
1) detecting the total number of lymphocytes in the sample,
2) detecting the percentage of the T lymphocytes to the lymphocytes in the sample; and
3) calculating the number of the T lymphocytes in the sample.

3. The immunophenotyping method according to claim 2, wherein step 2) comprises mixing a composition 2 comprising antibodies labeled by different fluorescent labels with the sample, incubating, performing detection by flow cytometry, collecting detection data, and analyzing the detection data;
wherein, the composition 2 consists of an anti-CD45 antibody, an anti-CD3 antibody, an anti-CD4 antibody, and an anti-CD8 antibody.

4. An immunophenotyping kit for identifying a subset of T lymphocyte, consisting of antibodies labeled by different fluorescent labels, wherein the antibodies are an anti-CD45 antibody, an anti-CD3 antibody, an anti-CD4 antibody, an anti-CD8 antibody, an anti-CD25 antibody, an anti-CD127 antibody, an anti-CD45RO antibody, an anti-CD197 antibody, an anti-CD28 antibody, and an anti-CD95 antibody;
wherein, the anti-CD45 antibody is labeled with a fluorescent label of FITC:
the anti-CD3 antibody is labeled with a fluorescent label of APC-Cy7:
the anti-CD4 antibody is labeled with a fluorescent label of Percp-cy5.5 or BV650;
the anti-CD8 antibody is labeled with a fluorescent label of BV510;
the anti-CD25 antibody is labeled with a fluorescent label of BV421;
the anti-CD127 antibody is labeled with a fluorescent label of Alexa Fluor 700;
the anti-CD45RO antibody is labeled with a fluorescent label of BV785;
the anti-CD197 antibody is labeled with a fluorescent label of PE:
the anti-CD28 antibody is labeled with a fluorescent label of BV605;
the anti-CD95 antibody is labeled with a fluorescent label of APC.

5. (canceled)
